(19) United States
(12) Patent Application Publication
(10) Pub. No.: US 2009/0075367 A1
(43) Pub. Date: Mar. 19, 2009

(54) Systems and methods for storing and monitoring, and for evaluating and ensuring the quality of cord blood

(76) Inventor: Bennett Liss, Malibu, CA (US)

Correspondence Address: DILWORTH PAXSON LLP, 1500 Market Street, Suite 3500 E, PHILADELPHIA, PA, 19102, US

(21) Appl. No.: 12/148,731

(22) Filed: Apr. 21, 2008

Related U.S. Application Data
(60) Provisional application No. 60/913,131, filed on Apr. 20, 2007.

(51) Int. Cl. G06F 17/40; C12M 3/00

(52) U.S. Cl. 435/308.1; 702/187

(57) ABSTRACT

Processes and systems are provided for tracking, monitoring, receiving, documenting, and reporting conditions of a client's cord blood sample from collection through to storage. Processes and systems are provided for collecting or receiving data relating to the cord blood sample and reporting the data. The data may comprise procurement data, transportation data, and/or storage data relating to the cord blood sample. Processes and systems are provided to report a certification of the cord blood collection and storage processes.

### CROSS-REFERENCE TO RELATED APPLICATION

This application claims the priority of U.S. Provisional Patent Application Ser. No. 60/913,131 filed Apr. 20, 2007, entitled “Systems and Methods for Storing and Monitoring, and For Evaluating and Ensuring the Quality of Cord Blood”, the entirety of which is incorporated herein by reference.

### BACKGROUND OF THE INVENTION

1. Field of the Invention

The present invention relates to umbilical cord blood. More particularly, the invention provides systems and processes for tracking the locations of and monitoring conditions relating to cord blood collection, for evaluating and ensuring the quality of cord blood throughout a storage period, and for associating the skills and expertise of health care professionals with consultation about cord blood with the client and with ongoing care for cord blood during storage.

2. Background of the Invention

Cord blood, also known as “placental blood,” is the blood that remains in the umbilical cord and placenta following birth and after the cord is cut. Typically, cord blood is discarded along with the placenta and umbilical cord after delivery. Umbilical cord blood is a valuable source of stem cells, which are genetically unique to each baby and family, and which, according to research, have important properties for developing therapies and treatments for numerous medical conditions.

Stem cells are the body's master cells because they can differentiate into many other tissues, organs, and systems in the body. The stem cells found in cord blood are the building blocks of blood and the immune system. Stem cells typically reproduce to form red blood cells, white blood cells, and blood platelets. The three primary sources of stem cells are bone marrow, peripheral blood (the blood that circulates through your body), and umbilical cord blood.

The ability of cord blood stem cells to differentiate, or to change into other types of cells in the body, has recently been found to hold significant promise for improving the treatment of some of the most common diseases, such as, for example, heart disease, stroke, and Alzheimer's disease. This has led to the collection of cord blood for storage in public blood banks for use by the general population and private blood banks for use only by the donor and the donor's family. There can now be little doubt about the value to an individual, or the individual's family, of an available and viable supply of his or her own stem cells.

Umbilical Cord Blood (UCB) banking is a once in a lifetime opportunity that allows families to save or bank UCB stem cells following a baby's delivery. There are two basic types of umbilical cord blood banks, namely private banks and public banks. When UCB is saved or banked with a private bank, the UCB stem cells are uniquely preserved for the donor or the donor's family. There are families who believe they may have a potential medical need for stem cell therapy in the future and request that their baby's stem cells be privately banked and pay up to $5000 to do so. These families may use these stem cells for their immediate, and possibly extended, family members' use. Parents consider this private banking opportunity as a type of health insurance, or simply a sound investment providing comfort and confidence to their family. With private banking, the family retains the right to decide how and when the banked cells can be used. Banking provides a perception that the family maintains total control throughout the storage.

The goal when collecting cord blood is to obtain a maximum number of hematopoietic progenitor cells as is possible. The number of these cells is important to the success of a transplant or other therapeutic procedure. For example, a transplant recipient's time to neutrophil recovery is strongly correlated with the dose of cryopreserved, nucleated cells. A donor may be keenly interested in learning of the quality of the cells prior to paying fees for storage of the cord blood.

There are a number of problems associated with the cord blood storage industry. These problems can be generally summed up as an absence of educating the patient/client and an absence of notification of various critical information concerning the quality of a cord blood sample and any exposure of the cord blood sample to adverse conditions. These problems are played out during the pre-delivery consultations, the process of collection of the cord blood during birth and delivery, to cryopreservation at a processing and storage facility, and into long-term storage.

Under current practices and methodologies, a client is generally not notified of the quality of the cells collected from cord blood. In particular, the client is not notified of how many cells were collected and how this correlates to the future success of a prospective transplant or other therapeutic procedure. The client is also not notified of any adverse condition that the cord blood may have been exposed to, during and after collection through to storage.

These problems associated with a lack of notification of cord blood quality are further exacerbated by an industry trend of a near zero rejection rate of blood by private storage companies. Thus, some families are paying significant amounts of money to store non-viable, cord blood stem cells. In fact, other families are storing cells in an inferior manner and/or their cells are being subjected to abject environmental conditions without their knowledge. In short, inferior cord blood is being stored. This means that parents (consumers) are paying to collect and store cord blood that, in some cases, is unusable and should not be stored. This may be directly attributed to the absence of notification of cord blood quality.

Another clear problem area in the industry is the now ubiquitous problem of the absence of third party (independent) verification of the quality of the processing of a cord blood sample as it goes through the collection and storage process. Currently, there are no accountability mechanisms in place within the private cord blood storage industry to ensure consumer transparency of the quality of the cord blood on a per sample basis. Because there has been no examination and follow-up to the consumer by a qualified, unbiased third-party, consumers, who have purchased cord blood collection and storage services, as well as those who have not, have made uninformed decisions, or have made decisions based merely upon marketplace hype.

In particular, a health care professional has never been employed on a fee for service basis, or any other basis for that matter, to examine and approve cord blood quality associated with the collection, processing, transportation, and storage of a consumer's cord blood specimen, whether separately or collectively. In addition, a health care professional has never been employed on a fee for service basis or any other basis to vet cord blood storage facilities for the consumer, or to educate them on the differing policies, procedures, and protocols that differentiate one storage facility from another. Furthermore, there has been no tracking and/or monitoring systems or methods for collection and storage of a cord blood sample from delivery, to transit, to processing, to storage, and into long-term storage, either separately or collectively. Moreover, health care professionals have not been hired to examine data related to the conditions of a consumer's cord blood sample during long term cryopreservation. Finally, health care professionals have not been engaged with an information feedback mechanism, such as a web interface that tracks the consultation process of a patient within the Cord Blood Storage Industry sourced from the health care professional's personal patient records, or otherwise.

In any case, and at the very least, a client should receive material facts regarding the quality of their child's cord blood sample. These material facts are readily obtained, at a minimum, by way of viability testing and cell counting methods. With these results in hand, a client would be apt to making an informed decision about cord blood storage.

In summary, the problems in the industry include a lack of (a) verification to families that cord blood was properly collected, (b) notification to families about viability characteristics of drawn cord blood, (c) confirmation to families that cord blood arrived safely at a storage facility and was stored properly and securely, (d) notification of inferior storage of cord blood and exposure of cord blood to damaging environmental conditions, (e) accountability mechanisms in the cord blood storage industry, and (f) independent, third-party verification of a child's cord blood sample throughout the process of collection at birth to cryo-preservation in the storage lab to long-term storage.

Accordingly, there is a present need for systems and methods to address these problems. In particular, there is a present need for methods and systems configured to track, collect, and report various data concerning the quality of cord blood sample characteristics comprising cell viability and cell count, and any exposure of the cord blood sample to adverse conditions during the process of collection of the cord blood through to cryopreservation at a processing and storage facility and into long-term storage.

### SUMMARY OF THE INVENTION

The present invention meets these pressing needs of the cord blood collection and storage industry. In particular, the present invention provides processes and systems for tracking, monitoring, documenting, and reporting the location of and quality and conditions relating to a client's cord blood sample from collection through to storage and potential transplant for future medical needs.

A process is provided for reporting cord blood sample conditions. In one embodiment of the invention, the process may comprise the steps of collecting data that comprises at least one of procurement data, transportation data, and/or storage data relating to the cord blood sample. The process may further comprise the additional step of providing a report that presents at least one of the collection data, the transportation data, and/or the storage data. Systems for such collection and reporting is also provided.

A further process is provided for monitoring cord blood sample conditions. The process may comprise receiving data related to a condition of a cord blood sample wherein one or more of the sample conditions are monitored. The processes may also comprise generating a report where the report comprises data relating to the cord blood sample.

It is provided that each of the procurement data, the transportation data, and the storage data may comprise data relating to at least one temperature reading relating to the cord blood sample, at least one cell viability reading relating to the cord blood sample, at least one cell count relating to the cord blood sample, a time of procurement of the cord blood sample, a date of procurement of the cord blood sample, a volume of the cord blood sample, at least one pH reading of the cord blood sample, and/or other data relating to current best practices in the industry.

In another embodiment, the present invention may provide the further step of providing a certification of at least one of the collection data, the transportation data, and/or the storage data provided with the report.

In yet another embodiment, the present invention may provide the further steps of collecting cryopreservation data relating to a cryopreserved cord blood sample and providing a periodic report presenting cryopreservation data. The cryopreservation data may comprise at least one temperature reading relating to the cord blood sample in cryopreservation, at least one reading of cell viability of the cord blood sample obtained during cryopreservation, at least one reading of cell count of the cord blood sample obtained during cryopreservation, information relating to financial condition of a cord blood bank storing the cord blood sample, and/or other data relating to best practices in the industry.

In another embodiment, the present invention also may provide that the step of providing the report may occur at any time from cord blood sample collection, through transit, in processing, into storage, and during and even after storage.

In yet another embodiment, the present invention may also provide a system for reporting conditions of a cord blood sample. The system may comprise a procurement data tracker configured to obtain procurement data relating to the cord blood sample. The system also may comprise a transportation data tracker configured to obtain transportation data relating to the cord blood sample. The system may also comprise a further storage data tracker configured to obtain storage data relating to the cord blood sample. Finally, the system may comprise a reporter configured to provide a report having at least one of the procurement data, the transportation data, and/or the storage data.

In further embodiment, the present invention may also provide a system for monitoring cord blood sample conditions. The system may comprise a data receive that receives data related to the condition of a cord blood sample. The system may also comprise a report generator that generates reports that may comprise data related to a cord blood sample.

In another embodiment of the system of this invention, each of the procurement data, the transportation data, and the storage data comprises at least one temperature reading relating to the cord blood sample, at least one cell viability reading relating to the cord blood sample, at least one cell count relating to the cord blood sample, a time of procurement of the cord blood sample, a date of procurement of the cord blood sample, a volume of the cord blood sample, at least one pH reading of the cord blood sample, and/or other data relating to current best practices in the industry.

In yet another embodiment of the system, the reporter may be configured to provide procurement data, transportation data, storage data, and cryopreservation data at any time from cord blood sample collection, through transit, in processing, into storage, and during and even after storage.

In another embodiment, the present invention provides a system further configured to collect cryopreservation data for a cord blood sample and to provide a periodic report comprising at least cryopreservation data relating to a cord blood sample. In this embodiment, the reporter may be configured to provide cryopreservation data.

In yet another embodiment, the reporter may provide a certification of at least one of the collection data, the transportation data, and/or the storage data with the report at any time after the cord blood is first collected. The reporter may even provide a certification of the quality of the collection kit.

The present invention also provides a cord blood monitor kit comprising a cord blood sample transport bag and a tracking monitor configured to collect transportation data about a cord blood sample during shipment of the cord blood sample to a cord blood bank. The tracking monitor may be a temperature tracking monitor.

The present invention also provides a cord blood collection kit for collecting cord blood samples having a cord blood sample condition. The kit may comprise a cord blood sample transport container and a tracking monitor. The tracking monitor configured to collect data related to a cord blood sample.

The present invention also provides optional processes and systems for third-party certification and oversight. In particular, processes and systems are provided for health care provider, and possibly cord blood bank agent or representative, certification of the quality of cord blood sample collection, transport, processing, and storage conditions along with viability and cell count associated with a cord blood sample. The processes and systems of the present invention may also include reporting cord blood sample quality and any adverse exposure risks associated with the collection, transportation, and storage of the cord blood sample.

In one embodiment, the present invention may provide documented third-party oversight that accomplishes and is not limited to the following characteristics. In particular, the present invention may inform and educate consumers as to the pros and cons of private cord blood storage prior to making the decision to store their child's cord blood. The present invention also may inform and educate those consumers who have decided to store, what has occurred that could have adversely affected their child's priceless stem cells during the collection, transit, processing, and storage of their child's specimen, and also confirms for the consumer when the consumer's sample has maintained status quo conditions and characteristics. The present invention also may provide continual independent review of the consumer's stored sample throughout the years of storage. The present invention also may provide education of the health care professional. These features may be met by compensating health care professionals to review the data retrieved during the many stages involved in the tracking of the cord blood sample, specific to a particular consumer's stored sample. Furthermore, these characteristics may be met by creating a customized report specific to a particular sample for the health care professional to review and certify as accurate and complete.

### BRIEF DESCRIPTION OF THE FIGURES

FIG. 1 depicts an embodiment of the present invention that illustrates the general steps of the process of cord blood collection and storage;

FIG. 2 depicts an embodiment that illustrates a process of the present invention;

FIG. 3 is a diagram illustrating an embodiment of the present invention;

FIG. 4 depicts an exemplary cord blood data collection card of the present invention configured for use in collecting procurement data;

FIG. 5 depicts an exemplary identification form of the present invention configured for use in collecting procurement data;

FIG. 6 depicts an exemplary donor identification verification form of the present invention for use in confirming a donor's identity;

FIG. 7 depicts an exemplary cord blood collection form of the present invention for use in collecting procurement data;

FIG. 8 depicts an exemplary transportation log of the present invention for use in collecting transportation data;

FIG. 9 depicts an exemplary reception log of the present invention for use in collecting transportation data;

FIG. 10 depicts an exemplary cord blood processing form of the present invention for use in collecting storage data;

FIG. 11 depicts an exemplary freezing log of the present invention for use in collecting storage information;

FIG. 12 depicts an exemplary summary report of the present invention for use in presenting collection data, transportation data, and storage data;

FIG. 13 depicts an exemplary discrepancy report of the present invention for use in reporting deviation from any standard operating procedure for cord blood collection, transportation, or storage;

FIG. 14 depicts an exemplary cord blood storage facility report of the present invention;

FIG. 15 depicts an exemplary consumer relations information report of the present invention;

FIG. 16 depicts an exemplary cord blood collection kit of the present invention;

FIG. 17 depicts an embodiment of the present invention illustrating a doctor enrollment process;

FIG. 18 depicts an embodiment of the present invention illustrating a general sales cycle for doctors;

FIG. 19 depicts an embodiment of the present invention illustrating a sales force structure; and

FIG. 20 depicts an embodiment of the present invention illustrating a general sales cycle for a patient/client.

### DETAILED DESCRIPTION OF EMBODIMENTS OF THE INVENTION

Understanding of the present invention will be facilitated by consideration of the following detailed description of embodiments of the present invention taken in conjunction with particular exemplary embodiments as described in the accompanying description and figures. It is to be understood that the figures and descriptions of the present invention have been simplified to illustrate elements that are relevant for a clear understanding of the present invention.

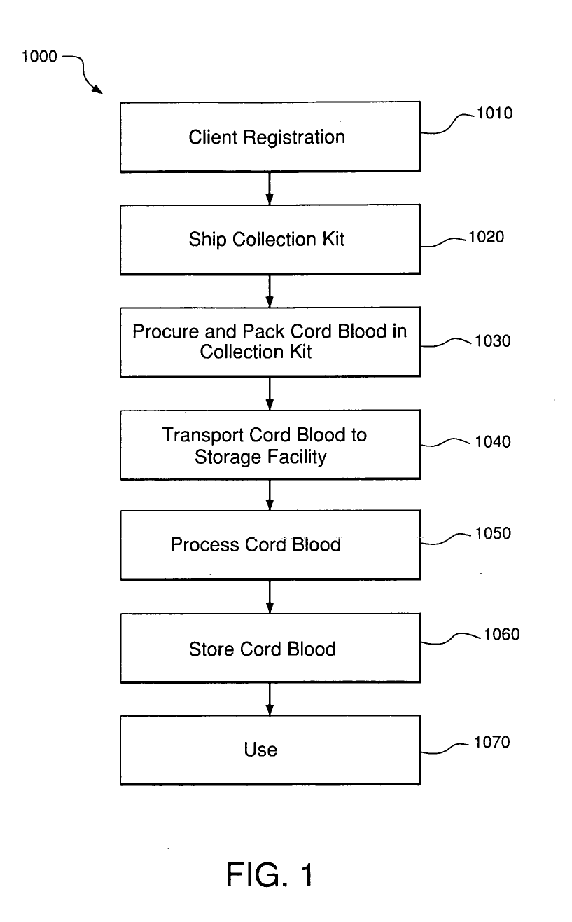

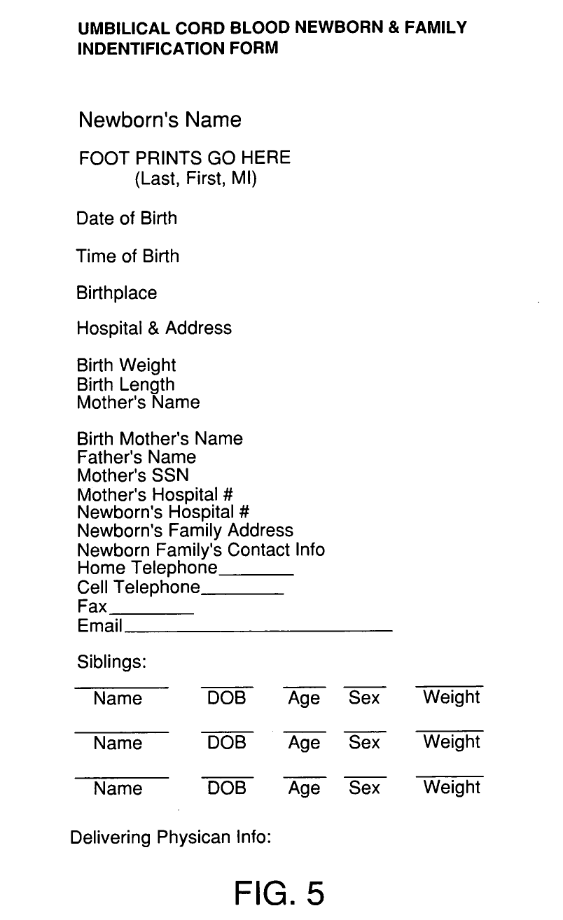

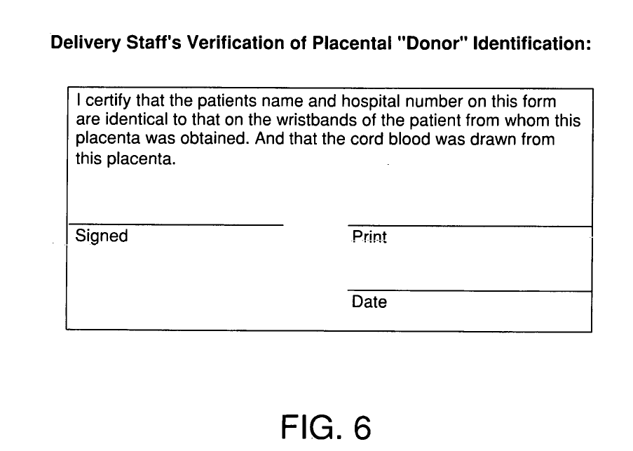

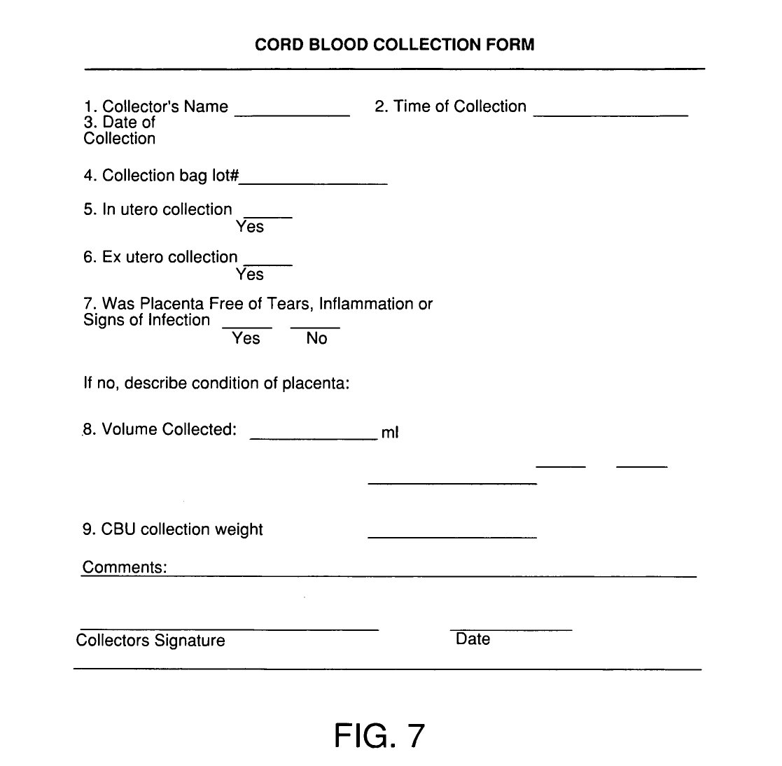

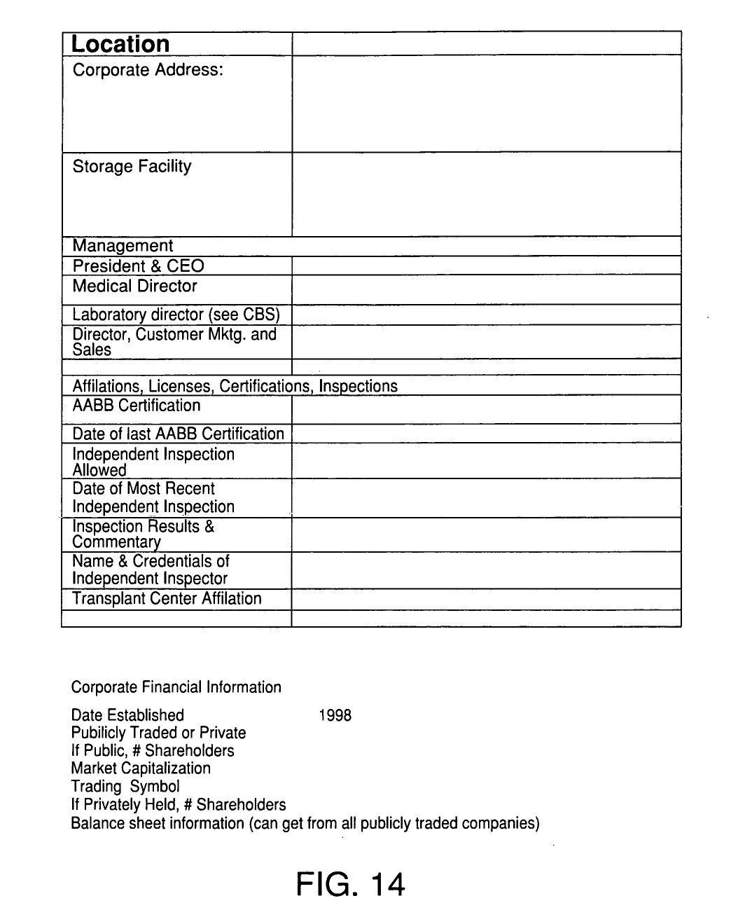

Referring now to FIGS. 1 through 20, the present invention provides processes and systems for collecting information, also referred to herein as data, relating to a cord blood sample. The present invention contemplates detailed collection and reporting protocols for the information, specifying data to be gathered and logged during and after collection of the cord blood, and identifying reporting and review procedures to be followed to verify specimen, collection, and storage characteristics as described hereinafter and illustrated by the figures hereto.

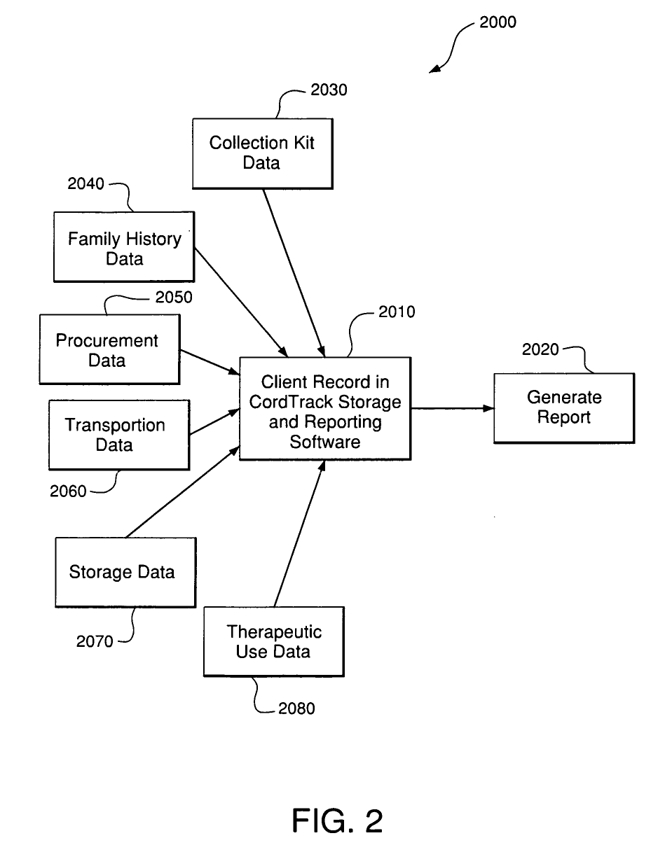

In an embodiment, and as illustrated in FIG. 2, the information relating to the cord blood sample may comprise procurement data, transportation data, storage data, therapy data, and any other data relating to a cord blood sample. From the time that a client becomes pregnant, information relative to cord blood, such as, for example, family history 2040, as depicted in FIG. 2, may be collected according to the processes and systems of the present invention. Other information relative to a cord blood sample may be collected from the time a client registers with the CordTrack service provider for cord blood collection and receives a collection kit 500. Further information may be collected in association with the procurement of a cord blood sample and transportation of the collected cord blood sample to a cord blood storage facility for cryopreservation. Information relating to storage of the cord blood sample and even therapeutic use of the cord blood sample may be collected.

The processes and systems of the present invention may be designed to collect this information through various technologies, such as, for example, electronic data submission through a web interface, email submission, paper submission by mail, or other means of data submission, such as wireless transmissions. In certain embodiments, the submission technology may incorporate data encryption. Whatever form of submission, such form may permit compiling the information into a computer program for analysis and report generation.

It will be further appreciated that computer systems, protocols, and tools exist to implement the unique web interface, including web page design tools, dynamic web page generators that may facilitate automatic queries to patient databases populated with patient information to generate dynamic web pages on the fly, web servers to host such application, integrated e-mail systems to automatically generated timed e-mail-based notifications, secure password-based login systems to ensure the security of confidential information such as patient record data, and web browsers that may permit ubiquitous access to the information available via the unique web interface of the present invention.

For example, and in various embodiments, the various technologies including protocols, platforms, and software that may used in connection with the systems and processes of the present invention may comprise HTTP and/or TCP/IP for web data transfer, SMTP for auto-generated email transfer, HTTPS & SSL for encryption and secure data transfer, SOAP for web services. In addition, these technologies may also comprise Microsoft Windows Server 2003 for an operating system, Microsoft IIS 6.0 as a web server, Microsoft SQL Server 2005 as a database server, Microsoft ASP.NET 2.0 as an application platform, and AJAX 1.0 as a UI platform. Other suitable technologies may be used in connection with the present invention.

The processes and systems of the present invention may also comprise reporting the compiled information relating to the procurement data, the transportation data, the storage data, and optionally the therapeutic use data. The processes and systems may be configured to report this information in a real-time report, a one-time Phase I report, and periodic Phase II report(s) as these various forms of reports are described in further detail herein.

Inherent to the processes and systems of the present invention is the facilitation of communication between a client, the client's health care professional, and the cord blood storage provider that is selected by a client to store the client's cord blood. The CordTrack service provider facilitates this communication by serving as an interface between the client, the client's health care provider, and a cord blood storage facility. A client's health care provider may also be involved throughout the practice of the processes and systems of the present invention. The client's health care provider may be a physician, obstetrician, midwife, or other health care provider associated with providing care before and during a client's pregnancy through to delivery. The cord blood storage facility may also be involved in recording information throughout the practice of the processes and systems of the present invention.

It is contemplated that the processes and systems of the present invention may be used from the moment that a woman finds out that she is pregnant. After the first determination of pregnancy, a mother may or may not be considering storing her future child's cord blood. Informed mothers may have already decided to store the cord blood while other mothers may not have enough information yet to make a final determination. Regardless of the circumstances, the processes and systems of the present invention promote interaction between the mother and the mother's health care provider. In certain circumstances during the first sonogram, a health care professional may consult with parents and present the options for cord blood storage at cord blood storage facilities. The health care professional may provide parents interested in considering storage of a cord blood sample information on cord blood storage facilities to aide them in their decision-making process.

Recently, several states have enacted legislation requiring some level of education of a pregnant patient regarding the benefits of banking cord blood. In certain states, such as, California, and Maryland, the department of health is encouraged to educate pregnant patients. In other states, such as Arizona, Michigan, New Mexico, Washington, and Wisconsin, a prenatal health care provider is either encouraged or required to provide a pregnant woman with information about collecting cord blood. The systems and processes of the present invention would facilitate legal compliance.

Turning now to FIG. 1, there is shown an embodiment of process 1000 of collecting, transporting, and storing a sample of cord blood in accordance with the present invention. In step 1010, the client enrolls or registers in the system of the invention. Once registered, a collection kit is shipped to the client in step 1020. Using the supplies provided in the collection kit, the cord blood specimen is provided and packed in the collection kit in step 1030. The specimen is then transported to the storage facility in step 1040. In step 1050, the specimen is processed and stored in step 1060. The specimen is then available for use in step 1070.

Turning now to FIG. 2, there is shown data collection system 2000. System 2000 storage and reporting software 2010 receives and/or stores the client record and registration data, collection kit data 2030, family history data 2040, procurement data 2050, transportation data 2060, storage data 2070, and therapeutic use data 2080. Storage and reporting software 2010 also may generate reports 2020, wherein reports 2020 may contain any stored data related to the specimens collected. Such reports may then be distributed to clients as described herein.

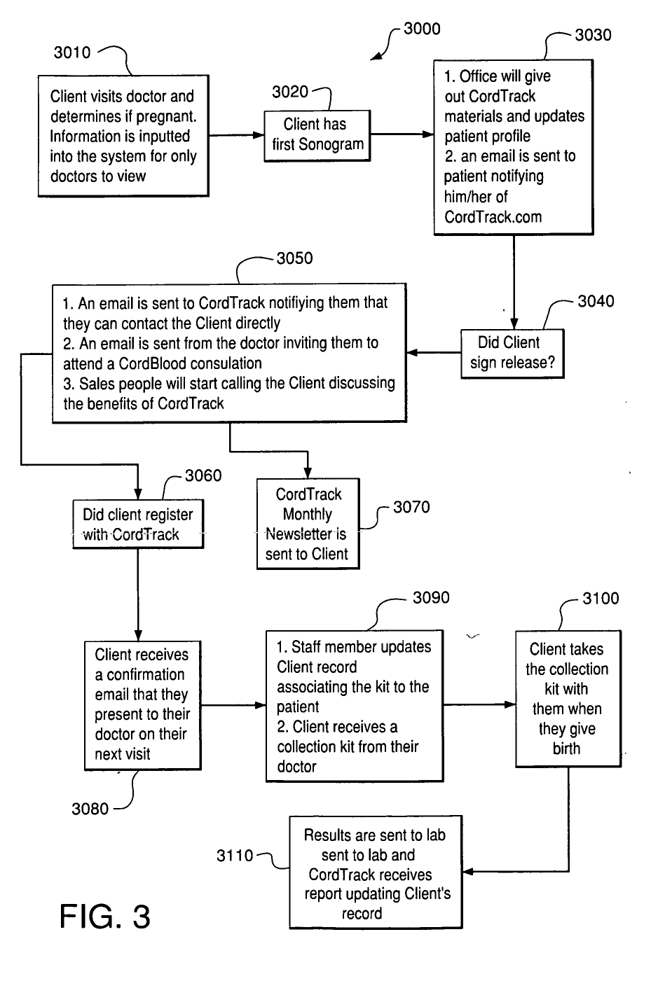

Turning now to FIG. 3, there is shown process 3000 for generating a report related to a client's cord blood specimen. In step 3010, the client visits the doctor to determine if the client is pregnant. Client data is entered into the system for doctors to view. The client has a first sonogram in step 3020. In step 3030, the doctor's office disseminates CordTrack material and updates the client profile and email can be sent to the client notifying the client of CordTrack services. In step 3040, there is a confirmation that the client signed a consent and release form. In step 3050, an email can be sent to CordTrack service provider with notice that the client may be contacted directly. An email can also be sent to the doctor's office with an invitation to attend a CordTrack consultation meeting. CordTrack sales representatives may contact the client to discuss the CordTrack cord blood service. In step 3060, there is a confirmation of whether the client registered. In step 3070, there may be a monthly newsletter sent to a client. In step 3080, an email confirming registration can be sent to the client so that the email can be presented to the client's doctor. In step 3090, the client's record is updated to associate the record with the client. The client can then receive the collection kit from their doctor. In step 3100, the collection kit is taken by the client to the facility for delivery and cord blood collection. In step 3110, the results obtained from the processed cord blood are reported to CordTrack service provider to update the client's record.

According to the present invention, in step 1010 depicted in FIG. 1, a client that decides to store cord blood will first register for the CordTrack service with the CordTrack service provider. At step 1010, client may enroll in the CordTrack service by completing and submitting an enrollment form through a secure web portal, or by mail or other courier. The enrollment form may be completed by providing information including, but not limited to, the name of client, the expected delivery date, the name of health care provider, and other relevant information. Once the enrollment form is completed and submitted, the CordTrack service provider will complete the registration process by entering a new record for the client into a computer program designed with data collection protocols to compile data relating to cord blood collection into electronic records and then report the cord blood data in various report formats.

Referring now to FIGS. 1 through 3, an embodiment of a system and process in accordance with the present invention is illustrated and implemented via a unique web-based interface. It will be appreciated that the steps illustrated in FIGS. 1 through 3 may be modified in nature, content, or number without departing from the essence of the unique and advantageous process flow identified therein. It will also be appreciated that the notifications illustrated therein may correspond to stages in a pregnancy, but may be altered without departing from the advantages of the process flow.

Referring again to FIGS. 1 through 3, the patient may complete a medical history some time prior to drawing of the cord blood. If the patient has not completed a medical history by a certain time prior to the estimated delivery date, the unique web-based interface of the present invention may advantageously generate as series of timed reminders by mail or email to a client. As another option, the patient may authorize providing access to all or part of the medical history information held by the patient's obstetrician or other health care professional.

In an embodiment, the present invention also includes a system and process, optionally web-based, which generates a unique code upon the reporting of the diagnosis of a pregnancy at a particular office or medical facility by a health care provider. The system notifies the CordTrack service provider of the new pregnancy, the unique code representing the new diagnosis and optionally the identification of a health care professional associated with the diagnosis. The CordTrack service provider may then, upon being notified of the newly diagnosed pregnancy, contact the health care professional and request that the health care professional notify the patient of the option of collecting and storing the cord blood. Advantageously, the notification may include the option presented to the patient to release patient identity and/or appropriate medical information from the patient's file to the CordTrack service provider (or other related cord blood service provider) for purposes of providing consultation and services related to collecting and storing the cord blood.

The computer program assigns a unique identification number to the client. The service provider provides the identification number to the client. The client's identification number is used through the processes and systems of the invention to identify the client, the client's cord blood sample and maternal peripheral blood reference sample, and other relevant information concerning the client. The client identification designation may be any numeric, alphanumeric, or other designation suitable for use to identify a client and distinguish the client from other clients registered with the service by service provider. The client identification designation may be used in support of HIPAA compliance. The client and/or the client's health care provider may use the identification number in accessing the CordTrack service provider's secure web portal.

The CordTrack service provider may also provide the client with a listing of cord blood storage facilities from which client may select one for processing and storage of the cord blood to be collected during delivery. The cord blood storage facilities may be certified by the CordTrack service provider as meeting its preset standards for cord blood collection, processing, and storage. These standards are designed to set a comprehensive analysis and certification for quality cord blood collection and storage. The standards may be synthesized from AABB, NMDB, FACT, and other cord blood industry standards. The CordTrack service provider, and optionally in conjunction with client's health care provider, may also provide the client with various educational materials relating to cord blood collection and cord blood-based therapies. These educational materials may facilitate state law compliance. This information may be provided to a client through the CordTrack service provider's secure web portal.

Once the client is registered in the service, service provider sends a collection kit to the client, or alternatively to the client's health care provider, as shown in FIGS. 1 and 3. Once the client goes into labor, the client, or alternatively the health care provider, brings the collection kit to the birthing facility. At the appropriate time during delivery, the health care provider collects a sample of cord blood. Once the sample is collected, the health care provider, or client, packages the cord blood sample in the collection kit and ships the collection kit with the cord blood sample to the designated cord blood storage facility.

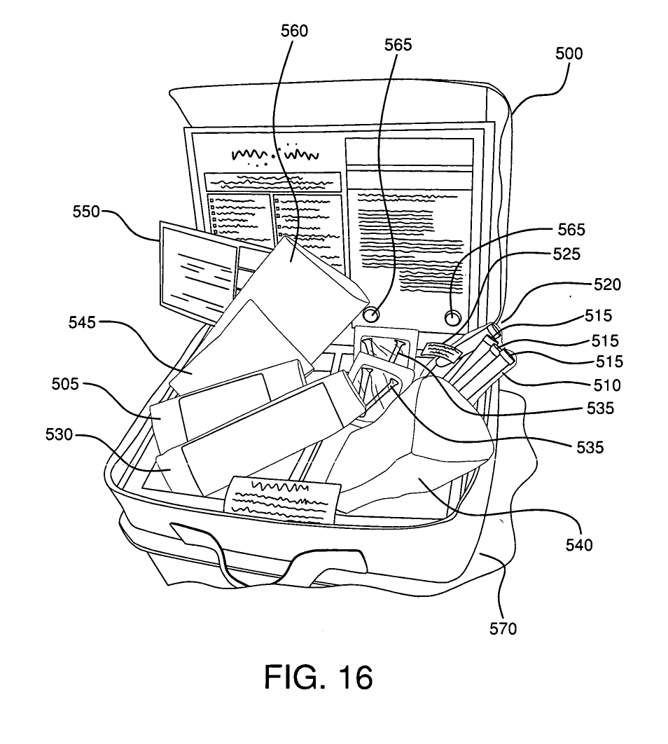

Referring to FIG. 16, the collection kit 500 may comprise a maternal blood specimen bag 505, a vial sleeve 510, three monoject plasma vials 515, a red serum vial 520, a monoject Magellan needle 525, an umbilical cord specimen bag 530, two hospital clamps 535, blood-pack unit (Baxter) 540, C-section adapter kit (C3-Centurion Scientific) 545, donor identification label 550, OB data collection card 555, leak proof storage bag for collected cord blood and maternal vials 560, at least one sensor to collect at least transportation data 565, and shipping pack 570.

According to the present invention, and once a client is registered at step 1010, the client or alternatively the client's health care provider may login to the service provider's Internet site and print out collection kit labels for use with identifying the client's cord blood sample. Alternatively, the collection kit labels may be included with the collection kit 500 and completed by a client or the client's health care provider prior to delivery. The collection kit labels may be used through the cord blood collection, transportation, and storage process to identify a client's cord blood sample.

The collection kit label may provide client information that may include the client's (mother's) name, unique client identifier, date of collection, time/time zone of collection, health care provider's name, designation of anticoagulant use, volume of total sample, name, city, state, and contact information for collection facility. The collection kit label may also include instructions for adhering the label to a blood bag and for shipment temperatures. In particular, the labels are placed on the inside and outside of the collection kit 500. The labels may also be placed on each vile of the collection kit 500.

In cases where the collection kit 500 is shipped to a client's health care provider, the client may pick up the collection kit 500 after the health care provider places the collection kit labels on the relevant parts of the collection kit 500. Alternatively, and in cases where the collection kit is sent directly to the client, the client may place the collection kit labels on the relevant parts of the collection kit 500. The placement of the labels may occur before the client enters into labor. Once the client goes into labor, the client may bring the collection kit 500 to the birthing facility. As illustrated in FIG. 3, a client may bring the collection kit 500 at the time of delivery. If the patient for some reason does not, a cord blood collection technician may have available another collection kit 500, possibly a universal spare kit.

To assist in the monitoring and tracking of environmental conditions to which cord blood is exposed, the present invention contemplates the use of sensors, which may sense environmental characteristics such as, for example, temperature, pressure, humidity and/or light, and these sensors may be located in delivery rooms, collection kits, transport packages or vehicles, and short-term or long-term storage facilities. The sensors may be human readable or may transmit sensed conditions digitally, electronically, and/or wirelessly to computer-based data storage devices capable of receiving such signals, interpreting them as needed, and associating the periodically sensed conditions with each particular cord blood sample in appropriate proximity to the particular sensor and also recording the time and date of each periodically sensed condition. In certain embodiments, sensors may be polled on demand by computer-based devices, such as, laptops, computer tablets, desktop PCs, PDAs, or cell phones. Sensors used may be equipped to transmit data in accordance with wire-based or wireless protocols such as, for example, Bluetooth, or any of the 802.11 series protocols.

The information that may be tracked includes the temperature variance of the cord blood while in transit. Each blood collection kit 500 is particular to a consumer who has made the decision to store cord blood, and at least one sensor 565 may be placed in the cord blood collection kit 500. In some cases, the data collected from the sensor 565 may be included in any report generated by the processes and systems of the present invention.

There are two types of sensors that may be used. One sensor may track the temperature continually from the time that the client receives the collection kit 500 to the moment the blood arrives at the cord blood storage facility. Once the collection kit 500 arrives at the cord blood storage facility, the sensor may be removed and may be read by a scanning device, and the entire temperature history of the contents of the box may be available for the health care professional to review, confirm, and/or include in any report prepared by the processes and systems of the present invention. In an embodiment, the sensor and reader device may be a Kooltrak system described at www.kooltrak.com. The information on the www.kooltrak.com website is incorporated by reference in its entirety.

Another type of sensor contemplated may indicate if the collection kit 500 containing the coagulants and other chemicals and/or the cord blood has ever fallen above or below appropriate temperature parameters. The indication on the sensor may come from a colormetric change, or some other type of indicator that once placed in the box changes if there has been a temperature violation.

Referring now to FIG. 1, in step 1030 a cord blood sample is collected by the client's healthcare provider collecting a volume of cord blood that is acceptable within the industry in the cord blood collection bag also referred to herein as blood pack unit 540. The health care provider may also draw reference samples of blood from the mother into the plasma vials 515. Once the blood is drawn, collection kit labels are affixed to the containers with the cord blood sample and the maternal reference samples. The labeled containers are packaged in the shipping pack for direct shipment to the cord blood processing and storage facility selected by the client.

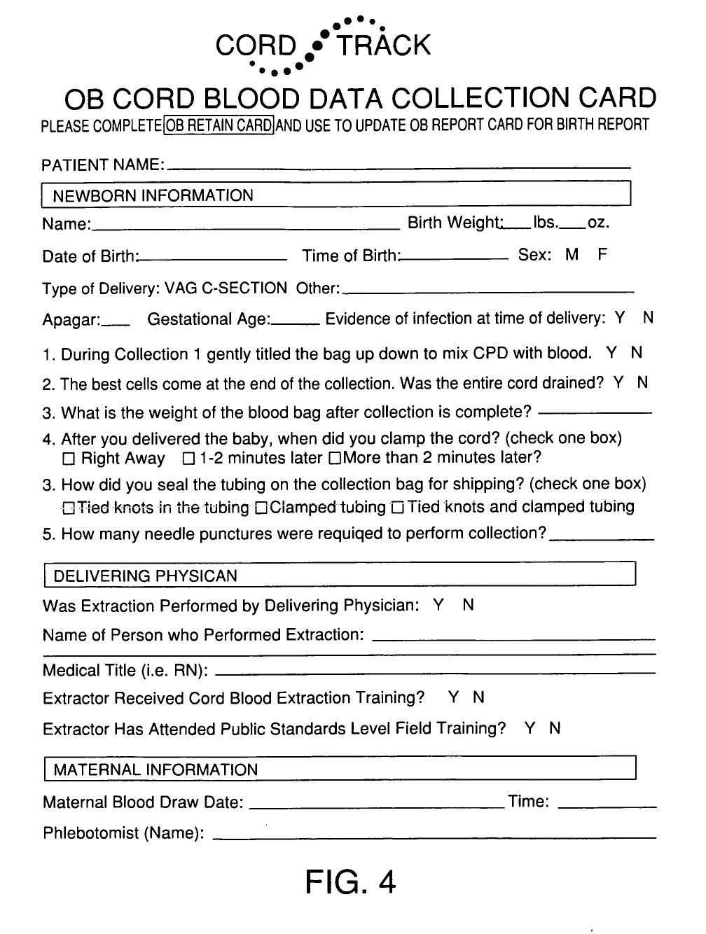

In an embodiment, the health care provider may complete the OB cord blood data collection card as shown in FIG. 4 provided with the collection kit 500 with procurement data. Procurement data may be initially recorded on an OB cord blood data collection card, an identification form, a donor identification form, a cord blood collection form as shown in FIGS. 4 through 7. The health care provider may use any of the procurement data recorded on an OB cord blood data collection card, an identification form, a donor identification form, a cord blood collection form the collection card to update the client's record through the CordTrack web portal. In an alternative embodiment, the client may input the procurement data on the collection card into the client's record in the CordTrack web portal.

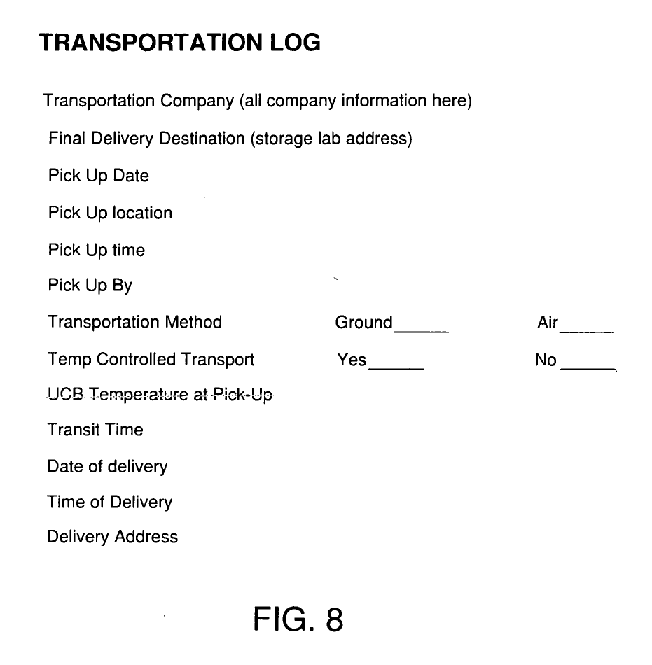

In step 1040 of FIG. 1, the client or client's designee provides the shipping pack 570 to a courier. The shipping pack 570 may contain at least a cord blood sample and a sensor, such as, for example, sensor 565. In an embodiment, the courier may be FedEx or other suitable carrier capable of handling shipment of biological materials. Sensor 565 may be used to collect transportation data relating to the cord blood sample throughout the course of shipment of the shipping bag to the cord blood storage facility. Additional transportation data may be compiled in a transportation log as shown in FIG. 8. The information in the sensor and the transportation log may be inputted into the client's record through the web portal by a client, the client's health care provider, or the client's cord blood storage facility. Optionally, the Cordtrack software program may interface with a courier's electronic shipping report for a collection kit that is accessible through the courier's website, and which the courier may send email updates and confirmation of the status of the shipment.

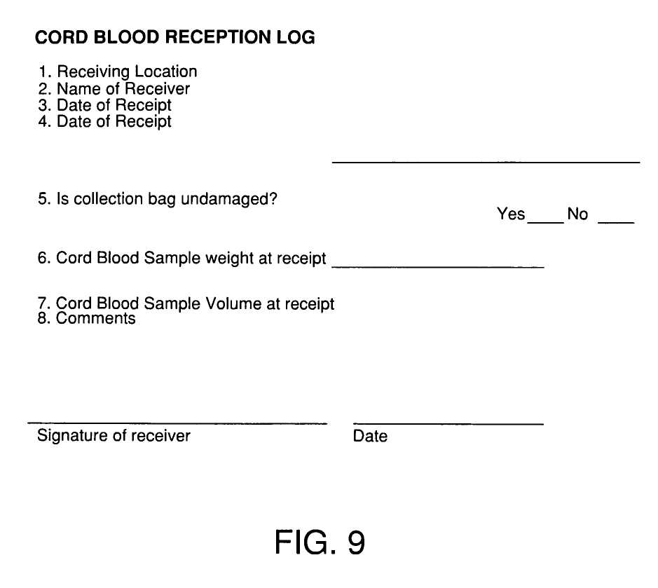

Referring now to FIG. 9, upon receipt of the cord blood sample in a shipping pack 570, the facility may complete a reception log with additional transportation data. The reception log may be completed with information relating to the receipt of the shipping pack 570 at the cord blood storage facility. This information may be saved by the facility to a client's record using the CordTrack web portal.

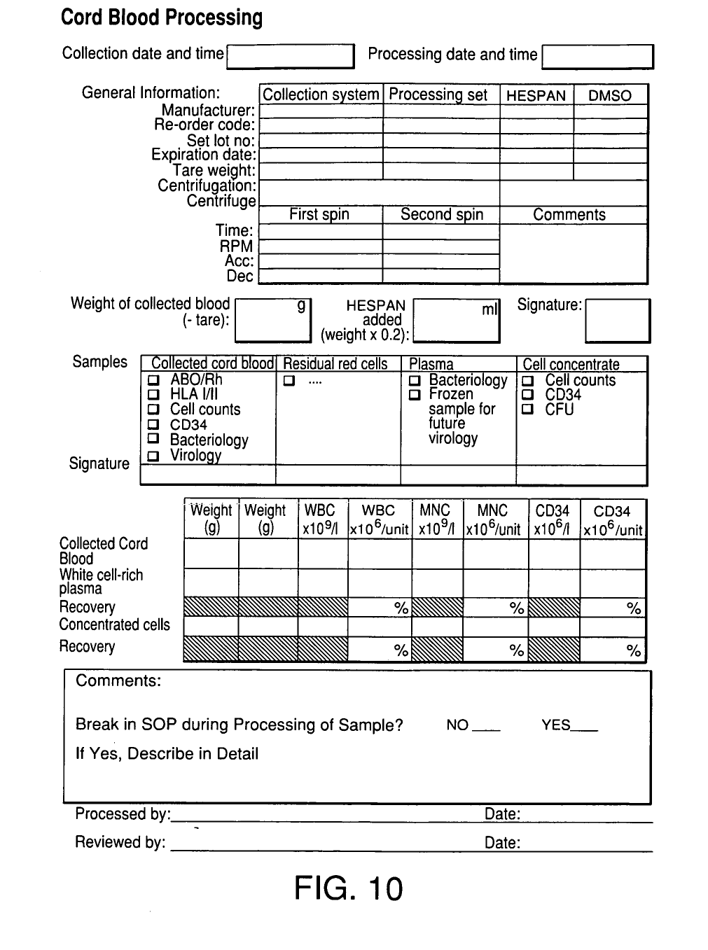

In step 1050 of FIG. 1, the cord blood storage facility will process the client's cord blood sample and maternal reference samples. During the course of processing the cord blood, the facility may record information concerning the cord blood sample, for example, the storage information referred to on the cord processing log in FIG. 10. This information may comprise cell viability and cell count for the cord blood sample. The information may be saved into client's record by the client, the client's health care provider, or the facility to the client's record on the CordTrack software through the CordTrack web interface. With this information stored in a client's record, the system may also generate a real-time report informing a client of the quality of the cord blood sample prior to committing the cord blood sample to storage. In such a case, the client may decide whether to store the cord blood sample or to have the storage facility discard the sample before it is stored.

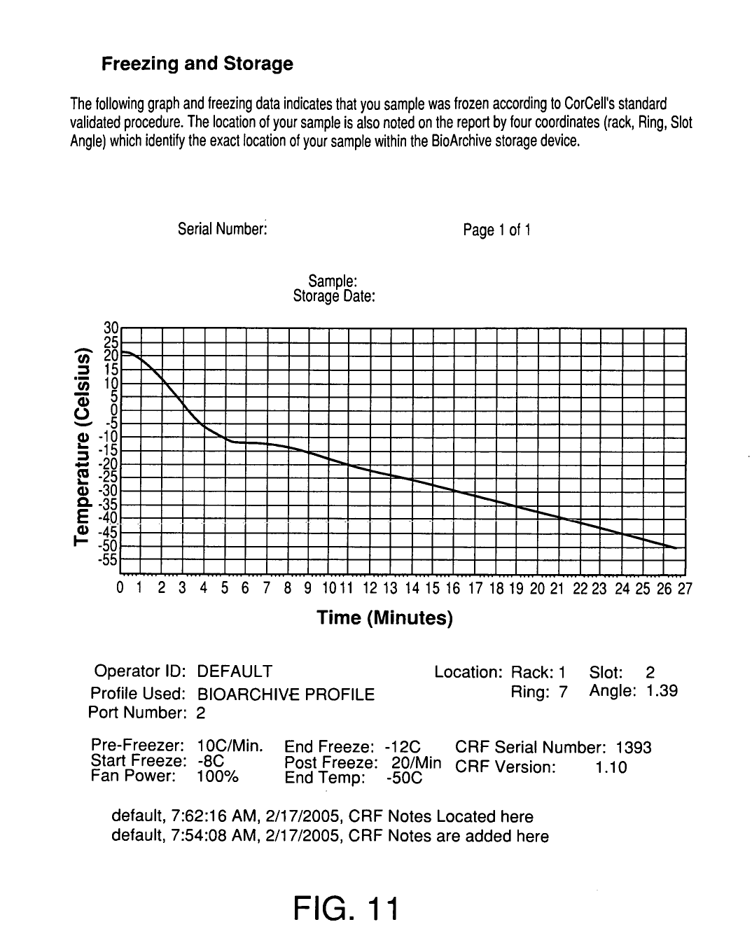

Additional information may be collected by the cord blood storage facility relating to the storage data. The facility may record cryopreservation information, such as, the information requested in FIG. 11. The facility or the client's health care provider may input the freezing information to the client's report on the CordTrack web portal.

The processes and systems of the present invention are configured to compile the information obtained from collecting data on the cord blood sample. The information may be reported in a real-time report. The real-time report may be configured to provide information collected at any point along the procurement, transportation, and storage process relating to the cord blood sample and as currently saved into a client's record accessible through the CordTrack web portal. The information may be reported in an initial Phase I report once the cord blood sample is stored. The Phase I report may be configured to provide all information collected from registration of the client in the CordTrack service up to storage of the cord blood sample.

The data may also be reported in periodic Phase II report(s) on the status of the cord blood in storage. A periodic Phase II report may be configured to provide storage information relating to storage of the cord blood sample and optionally use information relating to use of the collected stem cells. The periodic Phase II report may be configured to provide post-storage information pertaining to a cord blood sample that will be used in therapy or other application.

Upon compiling specific information retrieved from the cord blood sample collection process, data may be provided to a client's health care provider for review and approval. Once reviewed and approved, the CordTrack service provider may provide the initial Phase I report providing information detailing the cord blood's collection, what technique and equipment was used, who performed the collection and when, observations of anyone involved in the collection, thorough information about the cord blood itself, including viability, volume and cell counts, thorough patient information which may include anything observed and/or recorded about the patient by anyone on the delivery team, and environmental characteristics at all stages of the process from the delivery room to the storage facility and into storage.

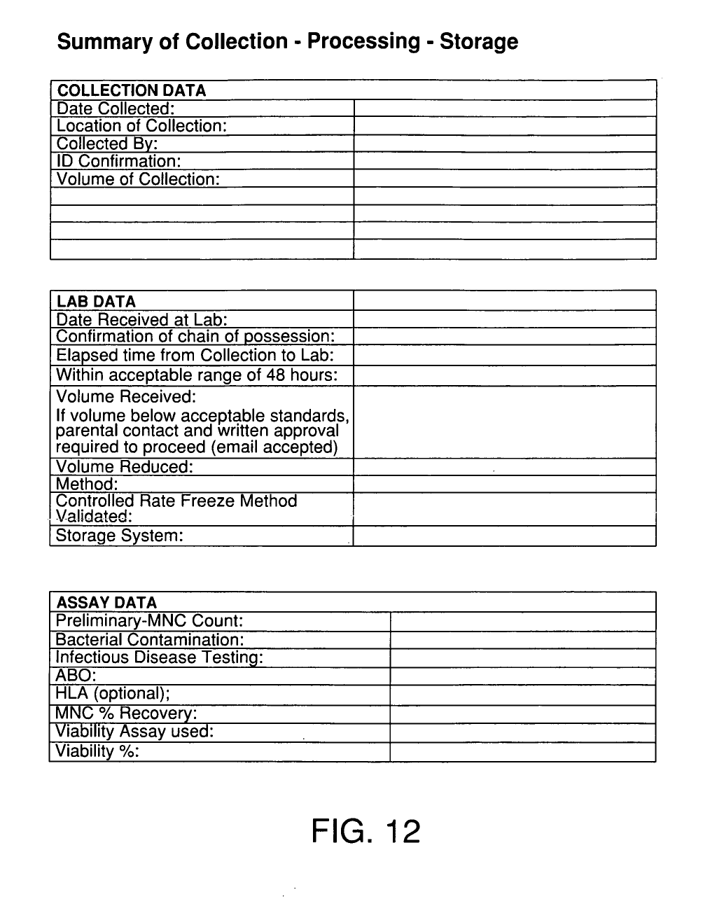

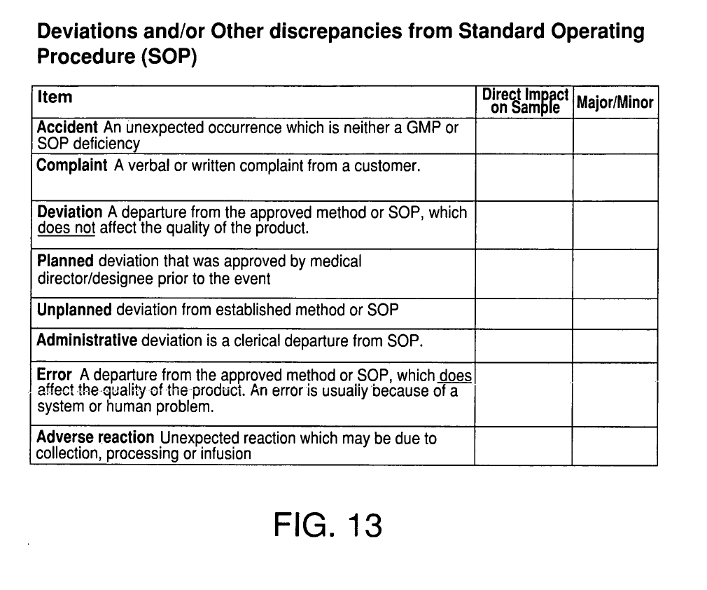

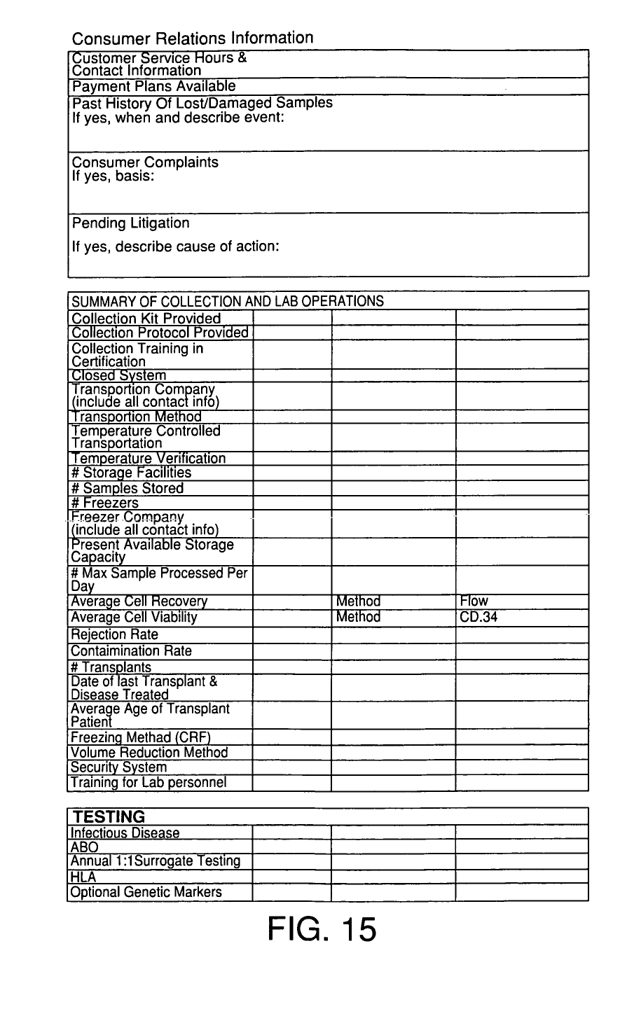

The Phase I report may include a compilation and presentation of any combination of data that may be recordable data presented in FIGS. 4 through 14. In addition, the Phase I report may comprise a summary presentation as generally illustrated in FIG. 12, a report regarding lack of compliance with standard operating protocols as illustrated in FIG. 13, and cord blood storage facility profile as illustrated in FIG. 15.

The health care provider's review, which may be a review by a licensed and board certified obstetrician, may verify or certify certain characteristics, including, but not limited to, the cord blood sample's viability, that it was collected, transported, and stored properly according to proper conditions, protocols, and procedures, and that the cell count met a minimum criteria for storage.

The information contained in the Phase I report may come from, but is not limited to, three sources, the patient, the cord blood storage facility, and the health care provider's records. Other information from other sources may include, for example, advances in stem cell therapeutics may also be included in the report. An initial Phase I report may be sent to the patient with the health care professional's signature attached.

In another embodiment, the Phase I report provided to the client may contain some other type of certification that the information has been reviewed, which certification may or may not supplement the signature of a health care professional. The Phase I report may be sent to the patient electronically or through regular acceptable postal communication, be it regular mail or special delivery through FedEx or some other postal entity.

After the initial Phase I report detailing the cord blood's journey from the delivery room to the storage facility and into storage, the processes and systems of the present invention enter phase II. In phase II, collecting information will continue specific to each client's sample while the sample is in cryopreservation. The continuing collecting of information may detail, but is not limited to, the ongoing operation of the storage facility, and may disclose any deviations in procedures, operations, or financial difficulties the storage company has undergone. It may also contain temperature or other variances the specimen has been subjected to while under storage. The health care professional, such as, for example, an obstetrician, may review the information periodically for a fee.

In one embodiment, the Phase II report may provide that proper medical protocols were followed during the time period covered by the cord blood collection process. The periodic report may also be approved/certified by a health care professional.

In some cases both the initial Phase I report and the periodic Phase II report will be on file, but may not yet be delivered to the consumer. At other times, both or one or the other of the Phase I report and the Phase II report may be delivered to the client.

Another unique aspect of the business process of the present invention is a proprietary web interface, as referenced throughout the disclosure, that is available to the client and the client's health care provider and accessed online. The process may track the client from the moment of pregnancy diagnosis or thereafter. This unique tracking, or collection of data relating to a cord blood sample, has been designed to include monitoring the client's consultation process. This patient tracking documentation process is unique and has never been available under the auspices of a health care professional working with cord blood collection and storage.

While it will be appreciated that the precise steps may vary in number, content or order, it will also be appreciated that this process may advantageously protect health care professionals and/or cord blood storage technicians and facilities, for example, by allowing them to document and evidence at later times that proper consultation had been provided by them to consumers who were considering cord blood storage options.

The CordTrack service provider may also advantageously offer the client the option to make a final decision on cord blood storage at a time following delivery. The presentation of the cord blood quality and the option to store or not to store is unique to the cord blood storage industry. The CordTrack service provider may obtain from the client permission to collect the cord blood, and would go forward and collect the cord blood and ensure proper storage of the collected blood for as long as the client desires prior to making a final decision. The client may agree to compensate CordTrack service provider in some amount for providing this option.

Moreover, the systems and processes of the present invention provide notification of the quality of a cord blood sample through its data receiving and reporting capabilities. The systems and processes may also be used to offer a guarantee as to the downstream success of a cord blood sample that was collected, transported, and stored in a manner that meets the certification process of the CordTrack service provider.

The client's health care provider and/or their staff may be compensated for functions relating to recording information about the cord blood sample, preparation of a Phase I and/or Phase II report, and assisting in the use of a stored cord blood sample. In an embodiment, payments from CordTrack service provider or another third party to the health care professional may be based on CordTrack service provider's maximum allowed time spent by the health care professional and the health care professional's staff on services that pertain to their role of gathering data for inclusions in any report and reviewing any report. The functions are initially performed, but not limited to, three distinct points, pre-delivery, delivery, and post-delivery. Additionally, the Phase II report may be reviewed by the health care professional.

In an embodiment, compensation for the health care professional, may be based, in whole or in part, on (i) the services that may require the involvement of the health care professional and their staff, (ii) the time involved in performing each service, and (iii) the current compensation levels of comparable health care professionals and various office staff positions. Compensation may also be based upon, or in part dictated by, other factors such as, for example, certain training or equipment.

If the client chooses to collect a cord blood sample at birth to be stored at a cord blood storage facility and utilize the services of a CordTrack service provider (or a related organization), the health care professional may take a detailed family history for any report prepared by the processes and/or systems of the present invention. The information collected by the health care professional may be uploaded to CordTrack software for later inclusion in any report which may be provided to the parent/patient. Prior to delivery, the health care professional may document his or her consultations with parents/patients for inclusion in any report, and the CordTrack service provider may advantageously compensate the health care professional for the health care professional's time. The CordTrack service provider may also advantageously compensate the health care professional for collecting the parent/family medical history as it relates to any report. It is assumed that the data gathered by the health care professional will be compiled and transmitted by a medical records staff in the health care professional's office, but the process is not limited to such transmission which could be accomplished by other means. The CordTrack service provider may also advantageously compensate the health care professional for the time of their medical records staff.

Compensation may advantageously involve services associated with delivery. The services performed during delivery relating to any report that the health care professional may be compensated for, including the collection of data on the delivery and the transmission of the data collected to the CordTrack service provider for inclusion in any report generated in connection with processes and systems of the present invention. Various details of the delivery may be recorded by a registered nurse (or other technician or professional) and the CordTrack service provider may advantageously compensate the health care professional for the registered nurse's (or other technician's or professional') time. The information collected may be entered and transmitted by a medical records staff and the CordTrack service provider may advantageously compensate the health care professional for the medical records staff's time.

Compensation may also advantageously involve post-delivery services. After the delivery, all details related to the shipping up to the point of pick up by the shipping company may be recorded and transmitted to the CordTrack service provider for inclusion in a Phase I report. Once the CordTrack service provider has received all the information required to prepare the Phase I report, the CordTrack service provider may provide the Phase I report to the health care professional for review. Recording all the details of the shipping and handling and transmitting the information to the CordTrack service provider may be handled by a medical records staff in the health care professional's office and the CordTrack service provider may advantageously compensate the health care professional for the medical records staffs time. For review of the completed Phase I report, the CordTrack service provider may advantageously compensate the health care professional for the health care professional's time.

Additionally, compensation may also involve services related to the periodic Phase II report. The CordTrack service provider may provide the health care professional with a period annual update report that provides information related to the ongoing operation of the cord blood bank and the storage of the cord blood and that provides current updates to information that was previously provided in earlier reports including the initial Phase I report. The CordTrack service provider may advantageously compensate the health care professional for time spent reviewing this material.

In further embodiments of the invention, additional health care provider enrollment and CordTrack sales processes are provided to complement the previously discussed embodiments.

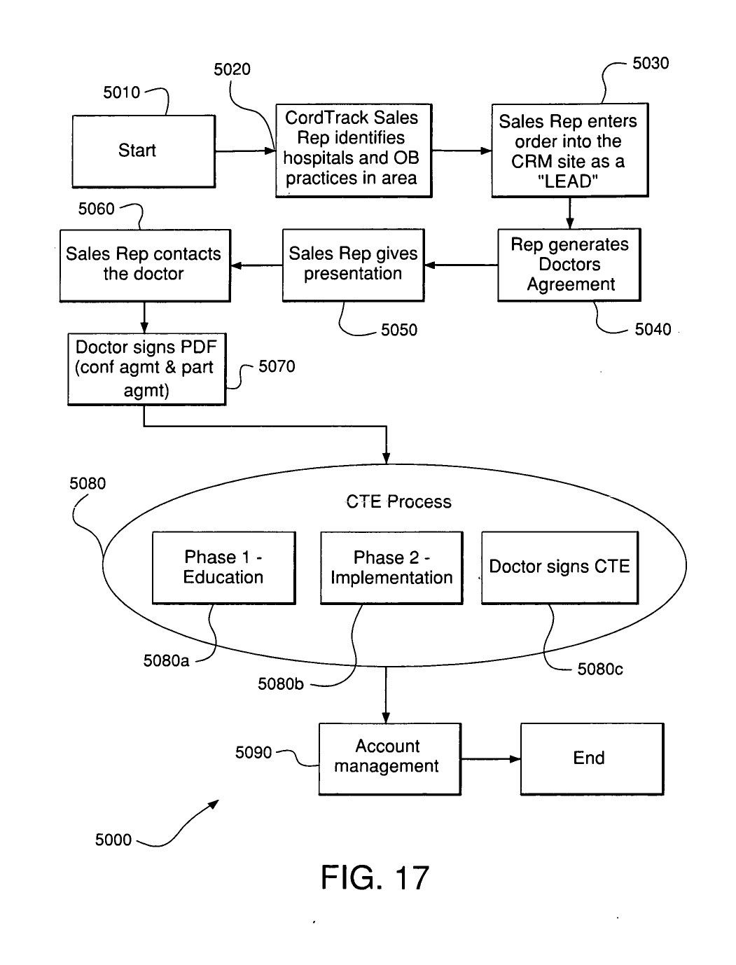

Referring now to FIG. 17, an embodiment of a health care provider enrollment process 5000 is depicted. In this exemplary embodiment, the enrollment process 5000 is initiated in step 5010. Once the process 5000 is initiated, a CordTrack sales representative identifies various hospitals and OB practices or other OB service providers in a particular area in step 5020. In step 5030, a health care provider is entered as a potential lead. The CordTrack sales representative contacts the health care provider in step 5040 and then gives a presentation about CordTrack to the health care provider in step 5050. If a health care provider decides to join the CordTrack service, the CordTrack sales representative provides an agreement to the health care provider wherein the agreement includes terms relating to the duties and obligations between the health care provider and CordTrack in step 5060. The health care provider enters into the agreement in step 5070. Once the agreement is executed and returned to CordTrack, the process enters into education of the health care provider regarding an execution plan of the CordTrack service, also referred to as CTE, in step 5080. The CTE process comprises Phase 1 education of the CordTrack service in step 5080a, Phase 2 implementation of the CordTrack service in step 5080b, and execution of the CTE agreement by a health care provider in step 5080c. Once the agreement is executed, the health care provider's account is opened and set up for management by a CordTrack representative in step 5090.

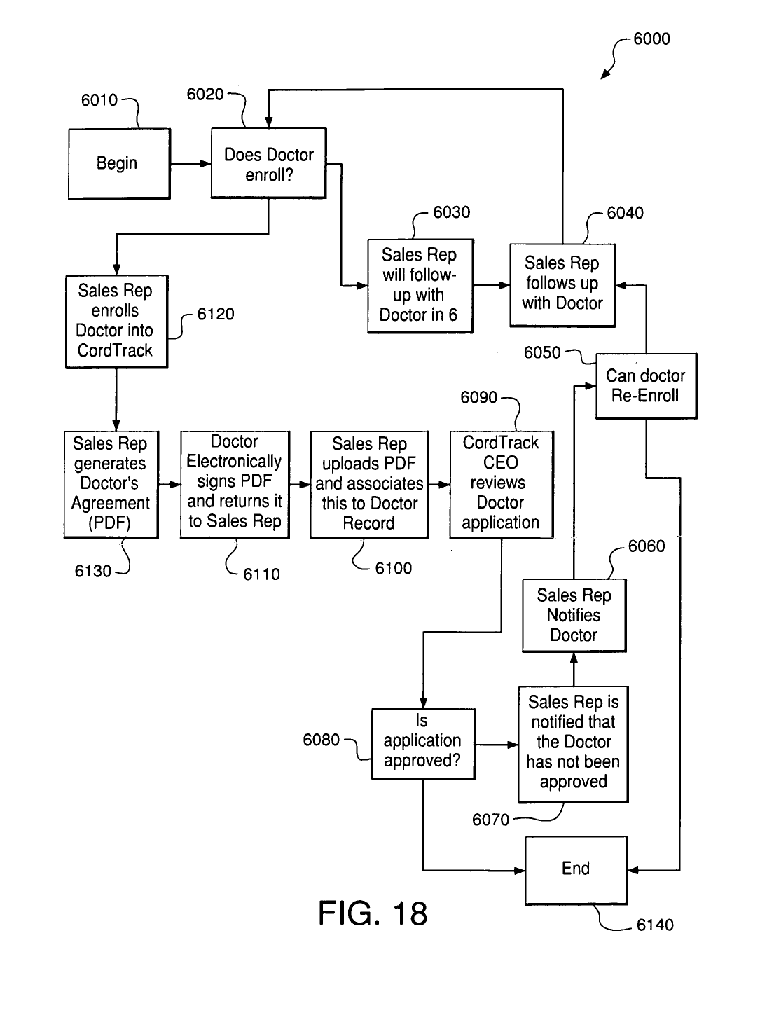

Referring now to FIG. 18, an embodiment of a sales cycle process 6000 for doctors of the present invention is illustrated. In step 6010, the sales cycle process is initiated. The sales cycle process comprises generally steps for enrolling the health care provider, steps for following up with health care providers that do not enroll, and steps for re-enrolling. In step 6020, a health care provider is presented with the option to enroll with CordTrack. If the health care provider decides to enroll, a CordTrack sales representative enrolls the health care provider in the CordTrack service in step 6120. The CordTrack sales representative generates an agreement for the health care provider in step 6130. The health care provider can execute the agreement and return it to the CordTrack sales representative in step 6110. The CordTrack sales representative can record the agreement into the health care provider's CordTrack record in step 6100. The CordTrack CEO or other authorized CordTrack agent can review the health care provider's agreement and application in step 6090. The health care provider can be approved or denied from enrolling in the CordTrack service in step 6080. If the health care provider is approved, this ends the health care provider sales cycle in step 6140.

Referring to FIG. 18, the health care provider can be denied in step 6070, and the CordTrack sales representative can notify the health care provider in step 6060. In step 6050, the health care provider has the option to re-enroll. If the health care provider elects not to re-enroll, the sales cycle process is completed in step 6140. If the health care provider elects to re-enroll in step 6050, the CordTrack sales representative will follow up with the health care provider in step 6040 and the health care provider can enroll in step 6020 and begin the enrollment process over again.

Referring to FIG. 18, the health care provider may decide to not enroll in step 6020. A CordTrack sales representative can follow up with the health care provider in about 6 months in step 6030. In step 6040, the CordTrack sales representative follows up with the health care provider.

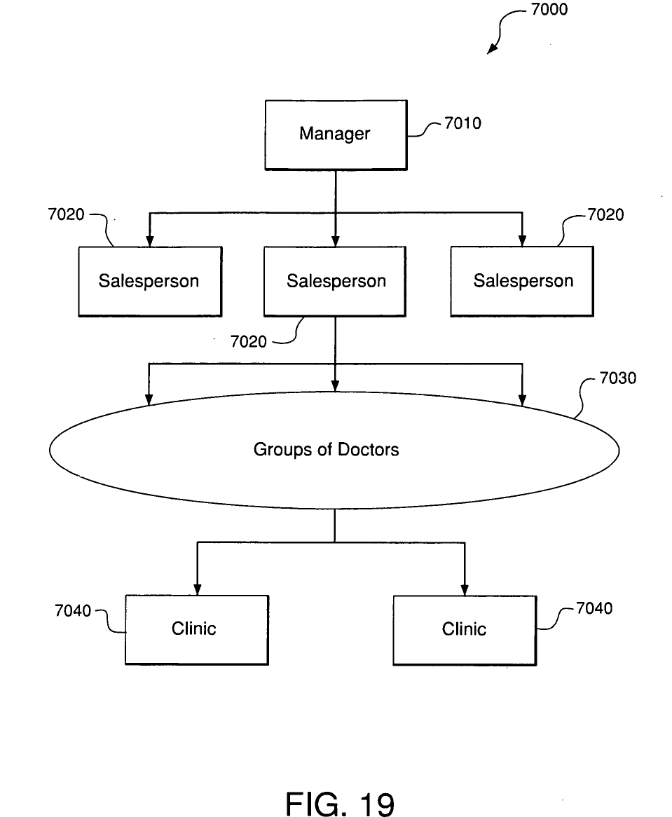

Referring now to a further embodiment of the present invention depicted in FIG. 19, an exemplary CordTrack sales force management process 7000 is illustrated. A CordTrack sales manager 7010 manages salespersons 7020 wherein the salespersons 7020 communicate with health care providers 7030 in there respective clinics 7040.

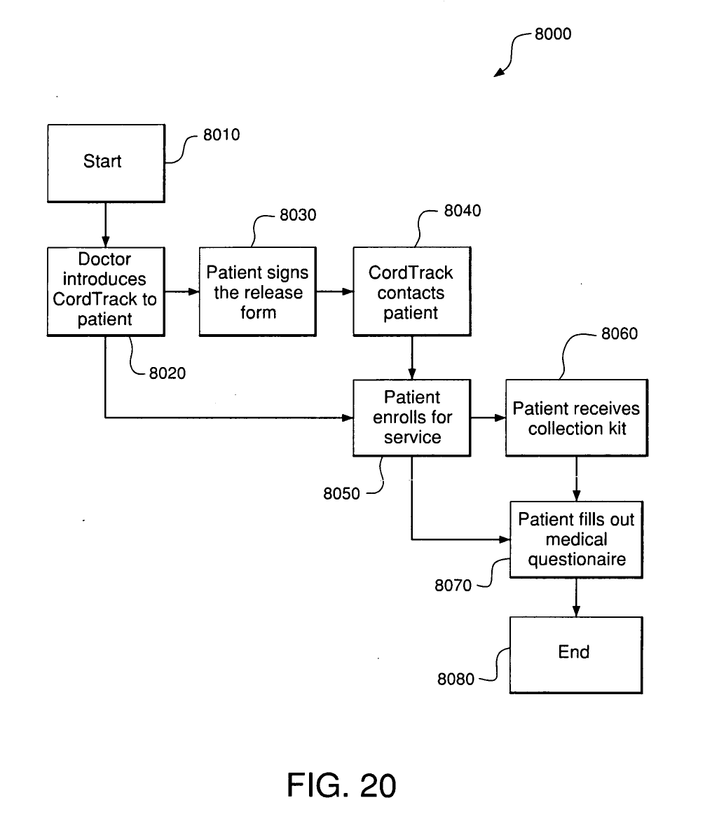

Referring to yet a further embodiment of the present invention depicted in FIG. 20, an exemplary CordTrack sales cycle system 8000 for a client is illustrated. The sales cycle is started in step 8010. In step 8020, a health care provider introduces the CordTrack service to a patient. The patient can enroll as a client in the CordTrack service in step 8050. Alternatively, the patient can sign a release form in step 8030 and a CordTrack sales representative can then directly contact the patient in step 8040. The patient can then enroll as a client in the CordTrack service in step 8050. In step 8060, a collection kit can be sent to a client. In step 8070, a client can complete a medical questionnaire after step 8050 or step 8060. The sales cycle is completed in step 8080.

Modifications can be made to the embodiments described above without departing from the broad inventive concept thereof. Having described the preferred embodiments of the invention, additional embodiments, adaptations, variations, modifications, and comparable arrangements will be apparent to those skilled in the art. These and other embodiments will be understood to be within the scope of the appended claims.

What is claimed is:

1. A process for monitoring cord blood sample conditions, said process comprising the steps of:
(a) receiving data related to said condition of said cord blood sample, wherein one or more of said sample conditions are monitored; and
(b) generating a report, said report comprising said data related to said cord blood sample.

2. The process of claim 1, wherein one or more of said cord blood sample conditions may be substantially continuously monitored.

3. The process of claim 1, wherein said sample conditions may be monitored during at least one of a period of time when: (i) said cord blood sample is collected; (ii) said cord blood sample is being transported; (iii) said cord blood sample is being processed; and (iv) said cord blood sample is being stored.

4. The process of claim 1, wherein said data comprises at least one of collection data, procurement data, transportation data, and storage data.

5. The process of claim 1, wherein said data comprises at least one temperature reading related to said cord blood sample.

6. The process of claim 1, wherein said data comprises at least one cell viability reading related to said cord blood sample.

7. The process of claim 1, wherein said data comprises at least one cell count related to said cord blood sample.

8. The process of claim 1, wherein said data comprises a time of procurement of said cord blood sample.

9. The process of claim 1, wherein said data comprises a date of procurement of said cord blood sample.

10. The process of claim 1, wherein said data comprises a volume of said cord blood sample.

11. The process of claim 1, wherein said data comprises a pH reading of said cord blood sample.

12. The process of claim 1, wherein said data comprises a location of said cord blood sample.

13. The process of claim 1, wherein said data comprises information related a facility storing said cord blood sample.

14. The process of claim 1, wherein said data comprises data relating to current best practices.

15. The process of claim 3, wherein said storage comprises cryopreservation of said cord blood sample.

16. The process of claim 1, further comprising the step of:
(c) receiving certification of said data.

17. A system for monitoring cord blood sample conditions, the system comprising:
(a) a data receiver which receives data related to said condition of said cord blood sample; and
(b) a report generator which generates reports comprising said data related to said cord blood sample.

18. The system of claim 17, wherein one or more of said cord blood sample conditions may be substantially continuously monitored.

19. The system of claim 17, wherein said sample conditions may be monitored during at least one of a period of time when: (i) said cord blood sample is collected; (ii) said cord blood sample is being transported; (iii) said cord blood sample is being processed; and (iv) said cord blood sample is being stored.

20. The system of claim 17, wherein said data receiver comprises a tracking monitor configured to collect data related to said condition of said cord blood sample.

21. The system of claim 20, wherein said tracking monitor is configured to collect temperature data related to said cord blood sample.

22. The system of claim 17, wherein said data receiver is configured to collect at least one of temperature, cell viability, cell count, time of procurement, date of procurement, volume, pH, geographic location, and current best practices data related to said cord blood sample.

23. The system of claim 17, wherein said data receiver is configured to collect information related a facility storing said cord blood sample.

24. The process of claim 19, wherein said storage comprises cryopreservation of said cord blood sample.

25. The system of claim 17, further comprising:
(c) a certification receiver which receives certification of said data.

26. A cord blood collection kit for collecting cord blood samples having a cord blood sample condition, said kit comprising:
(a) a cord blood sample transport container, and
(b) a tracking monitor configured to collect data related to said cord blood sample.

27. The cord blood collection kit of claim 26, wherein one or more of said cord blood sample conditions may be substantially continuously monitored.

28. The cord blood collection kit of claim 26, wherein said sample conditions may be monitored during at least one of a period of time when: (i) said cord blood sample is collected; (ii) said cord blood sample is being transported; (iii) said cord blood sample is being processed; and (iv) said cord blood sample is being stored.

29. The cord blood collection kit of claim 26, wherein said tracking monitor is configured to collect temperature data related to said cord blood sample.

30. The cord blood collection kit of claim 26, wherein said tracking monitor is configured to collect at least one of cell viability, cell count, time of procurement, date of procurement, volume, pH, geographic location, and current best practices data related to said cord blood sample.
